(19) United States
(12) Patent Application Publication
(10) Pub. No.: US 2006/0233738 A1
(43) Pub. Date: Oct. 19, 2006

(54) Composition for promoting production of type 1 collagen and/or elastin

(76) Inventors: Satoshi Miyata, Kanagawa (JP); Youko Handa, Kanagawa (JP); Tetsuhito Sakurai, Kanagawa (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/550,224

(22) PCT Filed: Mar. 23, 2004
(86) PCT No.: PCT/JP04/03978

§ 371 Date: May 2, 2006

(30) Foreign Application Priority Data
Mar. 25, 2003 (JP) 2003-083943

(51) Int. Cl. A61K 36/28; A61K 8/97; A61K 31/36

(52) U.S. Cl. 424/74; 424/764; 514/464

(57) ABSTRACT

This invention aims to provide a composition that promotes the production of type I collagen and/or elastin in the human skin fibroblast cells, wherein the composition improves the suppleness and elasticity of the skin, is amply effective in preventing and improving wrinkles and sagging, and is also very safe to the skin. The present invention relates to a composition that contains silymarin, which is a general term for flavonolignans such as silybin, silydianin, silychristin and isosilybin, wherein the aforementioned composition has a property to promote the production of type I collagen and/or property to promote the production of elastin. It also relates to a composition containing silymarin derived from a silymarin-containing plant and/or extract of such plant, wherein the aforementioned composition also has a property to promote the production of type I collagen and/or property to promote the production of elastin.

### TECHNICAL FIELD OF THE INVENTION

The present invention relates to a composition having the effect of promoting the production of type I collagen and/or elastin in the fibroblast cells derived from the dermis of human skin (hereinafter referred to as “human skin fibroblast cells”). More specifically, this invention relates to a composition that improves the suppleness and elasticity of the dermis and also prevents and improves the visible signs of skin aging such as wrinkles and sagging caused by aging or exposure to ultraviolet light.

### BACKGROUND TECHNOLOGY

Changes that occur in the skin due to aging of the skin, such as wrinkles and sagging, are known to be accelerated by physical and psychological stress caused by aging, exposure to sunlight, and so on. As the skin ages, epidermal cells and fibroblast cells that comprise the skin tissues will decrease and the blood vessels that supply substances needed to support the activity of these cells will also decrease. In addition, the extracellular matrix that retains the skin structure will also change. One significant change is the decrease and change in type I collagen, type III collagen and elastin in the dermis, or type IV collagen and laminin in the base membrane connecting the epidermal layer and dermal layer. These changes make the epidermis, dermis and base membrane become flat (Non-patent Literature 1). In particular, type I collagen and elastin, which are key constituents of the dermis, have significant bearing on the suppleness and elasticity of the skin. Change and decrease in these proteins are main causes of wrinkles and sagging.

In order to prevent the visible signs of skin aging as described above, various formulations have been developed that focus on the production of collagens and elastin. For example, agents that promote collagen production include a composition that contains retinoid and extract from buds of fagaceous plants (Patent Literature 1), a composition that contains extract from saussurea plants (Patent Literature 2), a composition that contains pueraria root extract (Patent Literature 3), a composition that contains extracts from plumbago zeylanicum and cyperus rotundus (Patent Literature 4), a composition that contains lotus sprout extract (Patent Literature 5), a composition that contains extract from epimedium plants (Patent Literature 6), and a composition that contains alpinia speciosa extract (Patent Literature 7).

As for compositions that increase elastin in the dermis, cosmetics formulated with elastin (Patent Literatures 8 and 9), an elastin-production promoting agent (Patent Literature 10), and agents that inhibit elastase, which is an elastin degrading enzyme (Patent Literatures 10 and 11) have been developed. From these literatures, it has been shown that promotion of the production of type I collagen and/or elastin in the human dermal tissue cells will suppress the visible signs of skin aging and also suppress or improve wrinkles and sagging.

Among the compositions that promote the production of type I collagen and elastin, retinoic acid and its derivative, retinol, are known (Non-patent Literatures 2 and 3). However, they are not suitable for practical use in general applications, because they cause skin irritation, present safety concerns (Non-patent Literature 4), and are chemically unstable.

As described above, compositions that increase the levels of collagen and elastin in the dermis have been developed for the purpose of preventing the visible signs of skin aging. However, none of them offer both safety to the skin and sufficient effectiveness in achieving the intended objective.

The present invention is based on the newly discovered physiological properties of silymarin on the human skin fibroblast cells that cause skin aging. Among the prior arts relating to silymarin, representative technologies are described in Non-patent Literature 5 and Patent Literatures 12 through 17 below. It should be noted, however, that the present invention is different from any of these prior arts.

Non-patent Literature 1: Utility of Cosmetics—Progress of Evaluation Technologies and Future Outlook, The Society of Cosmecit Chemists of Japan (Ed.), Theories, Chapter 1, Section 7, Cosmetics for Reduction of Wrinkles, p162-177, Mar. 31, 2001, First Print.

Non-patent Literature 2: Verani, J., et al., All-trans retinoic acid stimulates growth and extracellular matrix production in growth-inhibited cultured human skin fibroblasts. J. Invest. Dermatol., Vol. 94, No. 5, p717-723, 1990.

Non-patent Literature 3: Tajima, S., et al., Elastin expression is up-regulated by retinoic acid but not by retinol in chick embryonic skin fibroblasts, J. Dermatol. Sci., Vol. 15, p166-172, 1997.

Non-patent Literature 4: Zouboulis, C. C., Retinoids: Is there a New Approach?, IFSCC Magazine, Vol. 3, No. 3, 2000.

Non-patent Literature 5: Okuda, T., ed., Encyclopedia Natural Medicinal Substances, Hirokawa Shoten, Mar. 3, 1986.

Non-patent Literature 6: Wagner, H., et al., Arznein. Forsch, 18, 696, 1968.

Non-patent Literature 7: Wagner, H., et al., Arznein. Forsch, 24, 466, 1974.

Non-patent Literature 8: Tittel, G., et al., J. Chromatogr., 135, 499, 1977.

Non-patent Literature 9: Tittel, G., et al., J. Chromatogr., 153, 227, 1978.

Non-patent Literature 10: Quercia, V., et al., Chromatography in Biochemistry, Medicine and Environmental Research, Frigerio A. (Ed.), Elsevier Scientific Publishing Company, Amsterdam, 1988, p1.

Non-patent Literature 11: Lowry, O., et al., J. Biol. Chem., 193, 265, 1951.

Non-patent Literature 12: M. J. Barttek, et al., J. Invest. Dermatol., 58, 114, 1972.

Non-patent Literature 13: Cosmetics Handbook, Nikko Chemicals Co., Ltd., Nihon Surfactant Kogyo K.K., Toshiki Pigment Co., Ltd. (Ed.), 10. Promotion of Percutaneous Absorption, 3. Evaluation of Percutaneous Absorption, p. 607.

Patent Literature 1: Publication of Unexamined Patent Application No. 2001-278783

Patent Literature 2: Publication of Unexamined Patent Application No. 2001-316240

Patent Literature 3: Publication of Unexamined Patent Application No. 2001-348338

Patent Literature 4: Publication of Unexamined Patent Application No. 2002-29923

Patent Literature 5: Publication of Unexamined Patent Application No. 2002-29980

Patent Literature 6: Publication of Unexamined Patent Application No. 2002-53427

Patent Literature 7: Publication of Unexamined Patent Application No. 2001-316275

Patent Literature 8: Registration No. 3121957

Patent Literature 9: Publication of Unexamined Patent Application No. 2001-72571

Patent Literature 10: Publication of Unexamined Patent Application No. 2002-293747

Patent Literature 11: Publication of Unexamined Patent Application No. 2002-205950

Patent Literature 12: Publication of Unexamined Patent Application No. Hei 5-286864

Patent Literature 13: Patent No. 2948818

Patent Literature 14: Publication of Unexamined Patent Application No. 2000-169328

Patent Literature 15: Publication of Unexamined Patent Application No. 2000-169332

Patent Literature 16: Patent Application No. 2002-255448

Patent Literature 17: Publication of Examined Patent Application No. Hei 5-9406

Patent Literature 18: Publication of Examined Patent Application No. Sho 63-41396

### SUMMARY OF THE INVENTION

The present invention aims to provide a new composition that promotes the production of type I collagen and/or elastin in the human skin fibroblast cells. In addition, the present invention aims to provide a composition for preventing the visible signs of skin aging that improves the suppleness and elasticity of the skin, is amply effective in the prevention and improvement of wrinkles and sagging, and is also very safe to the skin.

After an extensive search of constituents that promote the production of type I collagen and elastin, the inventor found that silymarin and/or extracts from plants containing silymarin such as silybum marianum, as well as these plants themselves, have an excellent property to promote the production of type I collagen and/or elastin. The present invention is based on this discovery.

In other words, the present invention relates to the following:
1. A composition characterized by containing silymarin and having a property to promote the production of type I collagen and/or a property to promote the production of elastin.
2. The composition as described in 1 above, wherein the silymarin is an extract from silymarin-containing plant and/or derived from silymarin-containing plant.
3. The composition as described in 1 or 2 above, which is used for preventing skin aging.
4. The composition as described in any of 1 to 3 above, which is used for external application on the skin.
5. The composition as described in any of 1 to 3, which is a food.
6. The composition as described in 4 above, which contains 0.7% to 2.0% of silymarin as an active ingredient.
7. The composition as described in any of 1 to 3 above, which is a cosmetic.

### BRIEF DESCRIPTION OF THE DRAWINGS

FIG. 1: A drawing illustrating how silymarin promotes the production of type I collagen in the skin fibroblast cells.

FIG. 2: A drawing illustrating how silymarin promotes the production of elastin in the skin fibroblast cells.

FIG. 3: A drawing illustrating how silymarin promotes the production of type I collagen in a three-dimensional human skin mode.

FIG. 4: A drawing illustrating how silymarin promotes the production of elastin in a three-dimensional human skin mode.

FIG. 5: A graph showing the permeability of silymarin into the dermis of pig skin.

FIG. 6: A graph showing the measured depths of wrinkles.

FIG. 7: A graph showing the measured sizes of wrinkles.

FIG. 8: A graph showing the measured results of a skin elasticity test.

FIG. 9: A graph showing the results of a feeling evaluation.

Table 1: A table showing the numerical results on the property to promote type I collagen production as measured by image analysis.

Table 2: A table showing the numerical results on the property to promote elastin production as measured by image analysis.

Table 3: A table showing the numerical results on the property of each tested substance to promote type I collagen production, expressed with respect to full-length type I collagen.

Table 4: A table showing the numerical results on the property of each tested substance to promote elastin production, expressed with respect to full-length elastin.

Table 5: A table showing recipe A and comparative example B.

### BEST MODE FOR CARRYING OUT THE INVENTION

Silymarin (CAS No. 65666-07-1) is a general term referring to flavonolignans extracted from silibum marianum gaerin (CAS No. 84604-20-6), and a composition that contains, among others, silybin (CAS No. 22888-70-6), silydianin (CAS No. 29782-68-1), silychristin (CAS No. 33889-69-9) or isosilybin (CAS No. 72581-71-6) and is expressed by the molecular formula C25H22O10 (Non-patent Literature 5). In the present invention, these compositions that contain flavonolignans contained in silibum marianum extracts are also called “silymarin” just like they are in prior arts. Also, silymarin is a mixture of flavonolignans as described above, and the content of silymarin in plant extracts and plants can be measured using a method utilizing a spectrophotometer (Non-patent Literature 6), a method utilizing thin-layer chromatography (Non-patent Literature 7), and a method utilizing high-speed liquid chromatography (Non-patent Literatures 8 through 10). Among these measuring methods, 2,4-dinitrohydrazine analysis, which is a method based on measurement utilizing a spectrophotometer, has been reported to the German Pharmacopeia (monograph relating to fruits of silybum marianum) and is widely used. In the present invention, determination of the contents of the aforementioned constituents is carried out using the 2,4-dinitrohydrazine analysis method, by indicating equivalent silymarin contents in percent by weight.

Since ancient times, silymarin has been used in Europe in the prevention and treatment of liver diseases. Silymarin is also widely known as an anti-oxidant. Silymarin is a composition having beneficial properties for the skin, and some of the known applications of silymarin include a formulation for treating psoriasis and atopic dermatitis (Patent Literature 12), a composition containing a complex of flavonolignan and phosphatide, used to treat red spots, burns, dystrophy of the skin or viscous membrane, skin inflammation and other conditions, to prevent the visible signs of skin aging, and to protect the skin from irritations caused by external factors such as radiation, wind and sunlight (Patent Literature 13), a skin-permeating barrier enhancer (Patent Literature 14), a sebum secretion suppressant (Patent Literature 15), a composition that prevents the visible signs of skin aging by preventing and improving flattening of the skin (Patent Literature 16), and a cosmetic that prevents the visible signs of skin aging by utilizing an anti-oxidation property (Non-patent Literature 17). However, any useful properties of these compositions with respect to the human skin fibroblast cells have not been known. With regard to the present invention, the effective dosages of silymarin needed to sufficiently promote the production of type I collagen as well as the production of elastin in a three-dimensional skin model, as well as the effective dosage of silymarin needed to achieve sufficient permeation into the dermis of pig skin, were examined. As a result, these properties could be achieved to notable degrees when the silymarin content was 0.7% or more. Therefore, the present invention is characterized by an effective silymarin content of 0.7% or more when silymarin is to be externally applied to the skin.

Methods to isolate silymarin from silybum marianum fruits at high purities have been reported, such as a method that can isolate silymarin at a purity of 70 to 80% and another method that can isolate silymarin at a purity of 90 to 96% (Patent Literature 18). Normally, silymarin is extracted from silybum marianum seeds and fruits using ethanol, ethyl acetate, acetone, etc., spray-dried into a dry powder form, and sold commercially as extract materials. In the present invention, these commercially available silymarin products that have been prepared in the aforementioned manner can be used directly. It is also possible to use concentrated extracts of the silymarin constituents taken from silybum marianum, such as silybin, silydianin, silychristin and isosilybin, or compounds made by isolating and refining these constituents.

Plants containing silymarin that can be used in the present invention include leaves, stalks, buds, flowers, wood parts, barks and other parts exposed above ground, roots, tubers and other parts buried underground, and seeds, resins and all other parts of the plants.

Silymarin and silymarin-containing plants that can be used in the present invention may be in a directly dried form, or a solution prepared by dissolving dried silymarin or silymarin-containing plant in various solvents. For example, silymarin or silymarin-containing plants can be dissolved in water; alcohols such as ethanol and methanol; polyhydric alcohol such as propylene glycol and 1,3-butylene glycol; and organic solvents such as ether, acetone and ethyl acetate.

In the present invention, silymarin-containing plants that have been naturally dried, dried by hot air, freeze-dried or fermented can be utilized directly. If plant extracts are used in the forms of various formulations, those obtained by extraction, enrichment, powdering and other processes performed per the normally used methods can be utilized.

Silymarin-containing compositions based on the present invention promote the production of type I collagen and/or elastin that are key proteins comprising the extracellular matrix of the dermis, improve the suppleness and elasticity of the skin, and prevent and improve wrinkle formation and sagging caused by aging, exposure to ultraviolet light, and so on, thereby keeping the skin youthful looking.

Compositions containing silymarin, silymarin-containing plants and extracts from such plants as proposed by the present invention can be manufactured as cosmetics and other agents applied externally to the skin, or foods to be ingested orally.

In their application as cosmetics, silymarin, silymarin-containing plants and extracts from such plants can be used as cosmetic constituents directly, or by adding them to wheat germ oil or olive oil in order to manufacture cosmetics.

In their application as foods, silymarin, silymarin-containing plants and extracts from such plants can be used as foods directly, or by adding various nutritious constituents, or they can also be mixed with desired foods. For example, desired auxiliary agents such as starch, milk sugar, malt sugar, vegetable oil powder, cacao powder and stearic acid may be added to silymarin or any silymarin-containing plant or extract from such plant, after which the mixture can be processed, through conventional means, into a shape that is easier to ingest, such as granule, grain, tablet, capsule or paste, for use as a health supplement or functional food. In addition, silymarin, silymarin-containing plants and extracts from such plants can also be added to various foods, such as ham, sausage and other processed meat products; fish cakes (kamaboko and chikuwa) and other processed seafood products; or bread, confectionary, butter, powder milk and other fermented milk products. They can also be added to beverages such as water, fruit juice, milk and soda.

The effective content of silymarin, silymarin-containing plant or extract from such plant in the target composition can be selected and determined as deemed appropriate based on the preparation method, type of obtained formulation and other characteristics of the silymarin, silymarin-containing plant or extract from such plant, and not limited to any specific contents. However, if the composition is used externally on the skin, it is desirable that the equivalent silymarin content be adjusted to 0.7 to 2.0 percent by weight. If the composition is used as tablets, drink or other forms of food, the content should preferably be 0.001 to 20 percent by weight. Normally, a preferred equivalent silymarin content of plant or plant extract is 0.01 to 60 percent by dry weight.

Under the present invention, the effective dosage of silymarin, silymarin-containing plant or extract from such plant in the target composition can be determined as deemed appropriate based on the application channel, application schedule, type of obtained formulation and other characteristics. For example, silymarin can be taken by an appropriate dosage between 0.01 to 10 g a day all at once or over several times. In the case of a composition that contains a silymarin-containing plant or extract from such plant, it is possible to take an appropriate dosage between 0.1 to 25 g a day, in dry weight, all at once or over several times.

A composition used externally on the skin as prepared in accordance with the present invention may contain, if appropriate for the type of application, oils such as vegetable oils, hydrocarbons such as waxes, higher fatty acids, higher alcohols, silicones, anion surface active agents, cation surface active agents, amphoteric surface active agents, non-ionic surface active agent, preservatives, sugars, metal ion blockers, polymers such as water-soluble polymers, viscosity increasing agents, powders, UV absorbents, UV blocking agents, moisture keeping agents such as hyaluronic acid, aromatics, pH adjustment agent, and so on. They can also contain vitamins, skin activation agents, blood-circulation promoting agents, normal-bacteria controlling agents, active-enzyme removing agents, anti-inflammatory agents, skin whitening agents, disinfectants, and other medicinal or bioactive ingredients.

Examples of oils in the aforementioned application include camellia oil, oenothera tetraptera oil, macadamia nut oil, olive oil, rape seed oil, corn oil, sesame oil, jojoba oil, germ oil, wheat germ oil, glycerin trioctanate and other liquid oils; cacao oil, coconut oil, hardened coconut oil, palm oil, palm kernel oil, haze wax, haze wax kernel oil, hardened oil, hardened castor oil and other solid oils; and bees wax, candelilla wax, cotton wax, rice bran wax, lanolin, acetic acid lanolin, liquid lanolin, sugar cane wax and other waxes.

Examples of hydrocarbons in the aforementioned application include liquid paraffin, squalene, squalane and micro crystalline wax.

Examples of higher fatty acids in the aforementioned application include lauric acid, myristic acid, palmitic acid, stearic acid, oleic acid, linoleic acid, linolenic acid, docosahexaenoic acid (DHA) and eicosapentaenoic acid (EPA).

Examples of higher alcohols in the aforementioned application include lauric alcohol, stearyl alcohol, cetyl alcohol, cetostearyl alcohol and other linear alcohols; and monostearyl glycerin ether, lanolin alcohol, cholesterol, phytosterol, octyl dodecanol and other branched-chain alcohols.

Examples of silicones in the aforementioned application include dimethyl polysiloxane, methyl phenyl polysiloxane and other chain polysiloxanes; and decamethyl cyclopentasiloxane and other cyclic polysiloxanes.

Examples of anionic surface active agents in the aforementioned application include sodium laurate and other fatty acid salts; sodium lauryl sulfate and other higher alkyl sulfate ester salts; POE triethanol amine lauryl sulfate and other alkyl ether sulfate ester salts; N-acylsarcosinic acid salts; sulfosuccinic acid salts; and N-acylamino acid salts.

Examples of cationic surface active agents in the aforementioned application include stearyl trimethyl ammonium chloride and other alkyl trimethyl ammonium salts; benzalkonium chloride; and benzethonium chloride.

Examples of amphoteric surface active agents in the aforementioned application include alkyl betaine, amido betaine and other betaine surface active agents.

Examples of non-ionic surface active agents in the aforementioned application include sorbitan monooleate and other sorbitan fatty acid esters; and derivatives of hardened castor oil.

Examples of preservatives in the aforementioned application include methyl paraben and ethyl paraben.

Examples of metal-ion blockers in the aforementioned application include disodium ethylenediaminetetraacetate, edetic acid, sodium edetate and other edetic acid salts.

Examples of polymers in the aforementioned application include Arabian gum, tragacanth gum, galactan, guar gum, carageenan, pectin, agar, quince seed, dextran, pullulan, carboxymethyl starch, collagen, casein, gelatin, methyl cellulose, methylhydroxyporopyl cellulose, hydroxyethyl cellulose, sodium carboxymethyl cellulose (CMC), sodium alginate, carboxyvinyl polymer (CARBOPOL, etc.) and other vinyl polymers.

Examples of viscosity increasing agents in the aforementioned application include carageenan, tragacanth gum, quince seed, casein, dextrin, gelatin, CMC, hydroxyethyl cellulose, hydroxypropyl cellulose, carboxyvinyl polymer, guar gum, xanthan gum and bentonite.

Examples of powders in the aforementioned application include talc, kaolin, mica, silica, zeolite, polyethylene powder, polystylene powder, cellulose powder, inorganic white pigment, inorganic red pigment, mica coated with titanium oxide, talc coated with titanium oxide, mica coated with colored titanium oxide and other pearl pigments, and organic pigments such as red 201 and red 202.

Examples of UV absorbents in the aforementioned application include para-aminobenzoic acid, phenyl salicylate, isopropyl paramethoxy cinnamate, octyl paramethoxy cinnamate and 2,4-dihydroxybenzophenone.

Examples of UV blocking agents in the aforementioned application include titanium oxide, talc, carmine, bentonite, kaolin and zinc oxide.

Examples of moisture keeping agents in the aforementioned application include polyethylene glycol, propylene glycol, dipropylene glycol, 1,3-butylene glycol, 1,2-pentane diol, glycerin, diglycerin, polyglycerin, xylitol, maltitol, maltose, sorbitol, dextrose, fructose, sodium chondroitin sulfate, sodium hyaluronate, sodium lactate, pyrrolidone carboxylic acid and cyclodextrin.

Examples of medicinal constituents include vitamins such as vitamin A oil, letinol and other vitamin A substances; rivoflavin and other vitamin B2 substances; pyridoxine hydrochloride and other vitamin B6 substances; L-ascorbic acid, L-ascorbic acid phosphate ester, L-ascorbic acid monopalmitic acid ester, L-ascorbic acid dipalmitic acid ester, L-ascorbic acid-2-glucoside and other vitamin C substances; calcium pantothenate and other pantothenic acids; vitamin D2, cholecalciferol and other vitamin D substances; and α-tocopherol, tocopherol acetate, DL-α-tocopherol nicotinate and other vitamin E substances. Other examples include various extracts such as placenta extract, glutathione, saxifraga stolonifera extract and other skin whitening agents; royal jelly, fagus crenata extract and other skin activation agents; capsaicin, zingherone, cantharidis tincture, ichthammol, caffeine, tannic acid, γ-oryzanol and other blood-circulation promoting agents; glycyrrhitinic acid derivative, glycyrrhetinic acid derivative, azulene and other anti-inflammatory agents; arginine, serine, leucine, tryptophane and other amino acids; and normal-bacteria controlling agents such as maltose sucrose condensate and lysozyme chloride. In addition, chamomile extract, parsley extract, fagus crenata extract, wine yeast extract, grapefruit extract, lonicera japonica extract, rice extract, grape extract, hop extract, rice bran extract, loquat extract, cork tree bark extract, coix seed extract, sialid extract, melilot extract, birch extract, liquorice extract, peony extract, soapwort extract, dishcloth gourd extract, pepper extract, lemon extract, gentian root extract, beefsteak plant extract, aloe extract, rosemary extract, salvia officinalis extract, thyme extract, green tea extract, seaweed extract, cucumber extract, clove extract, carrot extract, marronnier extract, hamamelis virginiana extract, mulberry extract and various other extracts.

Examples of compositions used externally on the skin as proposed by the present invention can be used in various forms such as aqueous solution, oil, emulsion, suspended solution and other liquid forms; gel, cream and other semi-solid forms; or powder, granule, capsule, microcapsule, solid and other solid forms. Known methods can be used to prepare the material into any one of these forms and made into various types of formulations such as lotion, emulsion, gel, cream, ointment, plaster, cataplasm, aerosol, suppository, injection, powder, granule, tablet, pill, syrup and lozenge. As for the methods of use, the above formulations can be coated, attached or sprayed on various parts of the body or ingested by swallowing. Among others, compositions proposed by the present invention can be suitably made into lotion, emulsion, cream, ointment, plaster, cataplasm and aerosol forms.

Examples of cosmetics in the aforementioned application include skin lotion, skin milk, skin cream, face mask and other skincare cosmetics; makeup base lotion, makeup cream, liquid-, cream- or paste-type foundation and other makeup cosmetics; hand cream, leg cream, body lotion and other body cosmetics; and bath agents.

Next, the present invention is further explained by using examples. It should be noted, however, that the effects of the present invention are not limited to these test examples and examples.

### Test Examples

[Preparation of Compound Solution for Evaluation Test]

Dimethyl sulfoxide of biochemical reagent grade (DMSO manufactured by Wako Pure Chemical Industries), retinoic acid that has been shown to have a property to promote the production of type I collagen (all-trans-retinoic acid manufactured by Wako Pure Chemical Industries; used as a positive control) and soybean lecithin (manufactured by Wako Pure Chemical Industries; used as a negative control) were used. Each compound was dissolved in DMSO to produce a stock solution, and an appropriate amount of each stock solution was added to a culture solution and applied to cells. By following the same procedure, a stock solution was prepared by dissolving silymarin (silymarin group manufactured by Sigma-Aldrich) in the aforementioned DMSO, and an appropriate amount of the stock solution was added to a culture solution to check the effect of adding silymarin.

[Culturing of Normal Human Skin Fibroblast Cells]

Normal human skin fibroblast cells (hereinafter referred to as “NFB”), provided as CCD1059 (purchased from Dainippon Pharmaceutical), were cultured in a skin fibroblast cell culture medium (FGM manufactured by Sanko Junyaku) at 37° C. in a 5% CO2 incubator. FGM was prepared by adding a human skin fibroblast cell growth factor (1 μg/ml), insulin (5 mg/ml), gentamycin (50 μg/ml) and amphotericin B (50 μg/ml) to a base fibroblast cell culture medium. This test used cells whose passage number was 3 to 7.

[Evaluation of Silymarin's Property to Promote Type I Collagen Production]

NFB was suspended in FGM by adjusting the content to 5×104/ml, and 10 ml of the suspension was disseminated in each Ø90-mm culture dish and cultured until a 70% confluent was obtained. Next, 10 ml of FGM to which each compound had been added to each concentration was placed in each dish and cultured for 24 hours. The cultured supernatant was collected and processed in a centrifugal separator at 150 G for 5 minutes to remove floating cells. Then, the obtained solution was further processed in a centrifugal separator at 12,000 G for 30 minutes to remove cell fragments. Ammonium sulfate was added to a saturation at 80%, and the mixture was agitated overnight at 4° C. Then, the mixture was processed in a centrifugal separator at 15,000 G for 30 minutes to allow protein to precipitate, after which the supernatant was removed and dissolved in 20-mM Tris-HCl (pH 7.5). Following an overnight dialysis in 20 mM Tris-HCl (pH 7.5) at 4° C., 20 mM Tris-HCl (pH 7.5) was further added to produce a x20 concentrate, which was then used as a western blotting sample.

Protein in the sample was separated by SDS-PAGE and transferred to a nitrocellulose membrane. After the transfer, the nitrocellulose membrane was soaked in a blocking solution (a solution prepared by dissolving skim milk in PBS containing 0.1% polyoxy ethylene (20) sorbitan monolaurate so that the skim milk concentration becomes 5%) and blocked for a day at 4° C. The obtained substance was washed in a washing agent (PBS containing 0.1% polyoxy ethylene (20) sorbitan monolaurate), soaked in a primary antibody (polyclonal antibody to type I collagen (manufactured by Rockland) that had been adjusted to 500 ng/ml using a washing agent), and then kept for 1 hour in room temperature to cause reaction. The obtained substance was washed, soaked in a secondary antibody (horseradish peroxidase labeling anti-rabbit-immunoglobulin G that had been adjusted to 250 ng/ml by a washing agent), and then kept for 1 hour in room temperature to cause reaction. The obtained substance was washed, and then detected using ECL plus western blotting detection reagent (manufactured by Amersham Biosciences). The detected band was scanned using an image scanner (manufactured by Amersham Biosciences) and analyzed using image master software to produce numerical data.

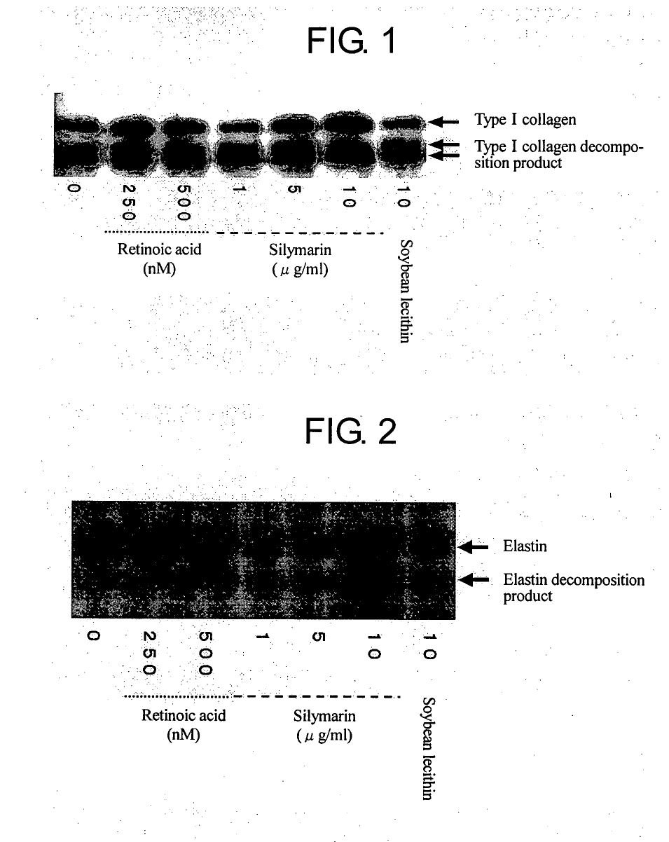

FIG. 1 shows the results measured via western blotting regarding the property of each compound to promote type I collagen production. Table 1 shows the numerical results obtained by image analysis with respect to full-length type I collagen (the band indicated as “Type 1 collagen” in FIG. 1).

TABLE 1

| Formulation | Treated concentration | | Ratio to untreated control |
|---|---|---|---|
| Untreated control | — | | 1.0 |
| Retinoic acid | 250 | nM | 2.0 |
| Retinoic acid | 500 | nM | 1.4 |
| Silymarin | 1 | μg/ml | 0.9 |
| Silymarin | 5 | μg/ml | 1.8 |
| Silymarin | 10 | μg/ml | 3.4 |
| Soybean lecithin | 10 | μg/ml | 0.9 |

When cells were treated with silymarin at a concentration of 5 and 10 μg/ml, production of type I collagen was promoted by 1.8 times and 3.4 times, respectively, compared with when the untreated control was used. When cells were treated with retinoic acid at a concentration of 250 nM and 500 nM used as a positive control, production of type I collagen was promoted by 2.0 times and 1.4 times, respectively, compared with when the untreated control was used. On the other hand, when cells were treated with soybean lecithin at a concentration of 10 μg/ml used as a negative control, the result was equivalent to when the untreated control was used. These results show that treatment of silymarin significantly promotes the production of type I collagen in the human skin fibroblast cells.

[Evaluation of Silymarin's Property to Promote Elastin Production]

NFB was suspended in FGM by adjusting the content to 5×104/ml, and 10 ml of the suspension was disseminated in each Ø90-mm culture dish and cultured until a 70% confluent was obtained. Next, 10 ml of FGM to which each compound had been added to each concentration was placed in each dish and cultured for 24 hours. The cultured supernatant was collected and processed in a centrifugal separator at 150 G for 5 minutes to remove floating cells. Then, the obtained solution was further processed in a centrifugal separator at 12,000 G for 30 minutes to remove cell fragments. Ammonium sulfate was added to a saturation at 80%, and the mixture was agitated overnight at 4° C. Then, the mixture was processed in a centrifugal separator at 15,000 G for 30 minutes to allow protein to precipitate, after which the supernatant was removed and dissolved in 20-mM Tris-HCl (pH 7.5). Following an overnight dialysis in 20 mM Tris-HCl (pH 7.5) at 4° C., 20 mM Tris-HCl (pH 7.5) was further added to produce a x20 concentrate, which was then used as a western blotting sample.

Protein in the sample was separated by SDS-PAGE and transferred to a nitrocellulose membrane. After the transfer, the nitrocellulose membrane was soaked in a blocking solution (a solution prepared by dissolving skim milk in PBS containing 0.1% polyoxy ethylene (20) sorbitan monolaurate so that the skim milk concentration becomes 5%) and blocked for a day at 4° C. The obtained substance was washed in a washing agent (PBS containing 0.1% polyoxy ethylene (20) sorbitan monolaurate), soaked in a primary antibody (polyclonal antibody to elastin (manufactured by Chemi-Con) that had been adjusted to 500 ng/ml using a washing agent), and kept for 1 hour in room temperature to cause reaction. The obtained substance was washed, soaked in a secondary antibody (horseradish peroxidase labeling anti-mouse-immunoglobulin G that had been adjusted to 250 ng/ml using a washing agent), and kept for 1 hour at room temperature to cause reaction. The obtained substance was washed, and then detected using ECL plus western blotting detection reagent (manufactured by Amersham Biosciences). The detected band was scanned using an image scanner (manufactured by Amersham Biosciences) and analyzed using image master software to produce numerical data.

FIG. 2 shows the results measured by western blotting regarding the property of each compound to promote elastin production. Table 2 shows the numerical results obtained by image analysis with respect to full-length elastin (the band indicated as “Elastin” in FIG. 2).

TABLE 2

| Formulation | Treated concentration | | Ratio to untreated control |
|---|---|---|---|
| Untreated control | — | | 1.0 |
| Retinoic acid | 250 | nM | 2.8 |
| Retinoic acid | 500 | nM | 1.4 |
| Silymarin | 1 | μg/ml | 0.9 |
| Silymarin | 5 | μg/ml | 1.1 |
| Silymarin | 10 | μg/ml | 4.7 |
| Soybean lecithin | 10 | μg/ml | 1.1 |

When cells were treated with silymarin at a concentration of 10 μg/ml, elastin production was promoted by 4.7 times compared with when the untreated control was used. When cells were treated with retinoic acid at a concentration of 250 nM and 500 nM used as a positive control, elastin production was promoted by 2.8 times and 1.4 times, respectively, compared with when the untreated control was used. On the other hand, when cells were treated with soybean lecithin at a concentration of 10 μg/ml used as a negative control, the result was equivalent to when the untreated control was used. These results show that treatment of silymarin significantly promotes the production of elastin in the human skin fibroblast cells.

[Evaluation of Silymarin's Property to Promote Type I Collagen Production in a Three-Dimensional Human Skin Model]

Three-dimensional human skin models are widely used in safety evaluations and effectiveness evaluations as pseudo human skins. In this evaluation, TESTSKIN (LSE-high) (manufactured by Toyobo) was used as a three-dimensional human skin model, and cultured in accordance with the protocol specified in the attached document. Silybum marianum extract containing 70% silymarin (manufactured by Indina), soybean lecithin (manufactured by Wako Pure Chemical Industries) and retinoic acid (all-trans-retinoic acid manufactured by Wako Pure Chemical Industries) were dissolved to respective concentrations in dipropylene glycol (manufactured by Wako Pure Chemical Industries). Silybum marianum extract containing 70% silymarin was dissolved in dipropylene glycol based on an equivalent concentration of the active ingredient silymarin. Silybum marianum extract containing 70% silymarin and soybean lecithin were heated before dissolution in dipropylene glycol, while retinoic acid was dissolved under agitation in room temperature. Dipropylene glycol was used as an untreated control.

Next, 60 μl of each tested substance was added to tissues in an assay ring and the tissues were cultured for 24 hours. Thereafter, the culture solution was changed and the assay ring was washed by the culture medium, after which a new sample was added and the tissues were cultured for 24 hours. The tissues were collected and mixed with a tissue extraction solution (50 mM Tris-HCL (pH 7.5), 0.5% (octylphenoxy) polyethoxy ethanol manufactured by Sigma-Aldrich) and homogenized using a Teflon homogenizer. The homogenized mixture was processed in a centrifugal separator at 10,000 G for 30 minutes to remove tissue fragments, after which it was dialyzed in distilled water overnight at 4° C. and then freeze-dried to remove water content. 20 mM Tris-HCl (pH 7.5) was added produce a x20 concentrate, which was then used as a western blotting sample. Protein in the sample was determined using a DC protein assay kit (manufactured by Bio-Rad Laboratories) based on the Lowry method (Non-patent Literature 11), and 20 mM Tris-HCl (pH 7.5) was added to achieve 2 μg/μl. The sample was mixed with an equal amount of x2 Laemmli sample buffer concentrate (manufactured by Bio-Rad Laboratories) containing 5% 2-mercaptoethanol (manufactured by Bio-Rad Laboratories) and heated at 100° C. for 5 minutes using a heat block to reduce the protein in the sample.

The obtained sample was used to evaluate the property to promote type I collagen production using the western blotting method. Specifically, 10 μg of protein was applied per lane, and after separation by SDS-PAGE the result was transferred to a nitrocellulose membrane. After the transfer, the nitrocellulose membrane was soaked in a blocking solution (a solution prepared by dissolving skim milk in PBS containing 0.1% polyoxy ethylene (20) sorbitan monolaurate so that the skim milk concentration becomes 5%) and blocked for a day at 4° C. The obtained substance was washed in a washing agent (PBS containing 0.1% polyoxy ethylene (20) sorbitan monolaurate), soaked in a primary antibody (polyclonal antibody to type I collagen (manufactured by Rockland) that had been adjusted to 500 ng/ml using a washing agent), and kept for 1 hour at room temperature to cause reaction. The obtained substance was washed, soaked in a secondary antibody (horseradish peroxidase labeling anti-rabbit-immunoglobulin G that had been adjusted to 250 ng/ml using a washing agent), and kept for 1 hour at room temperature to cause reaction. The obtained substance was washed, and then detected using ECL plus western blotting detection reagent (manufactured by Amersham Biosciences). The detected band was scanned using an image scanner (manufactured by Amersham Biosciences) and analyzed using image master software (manufactured by Amersham Biosciences) to produce numerical data.

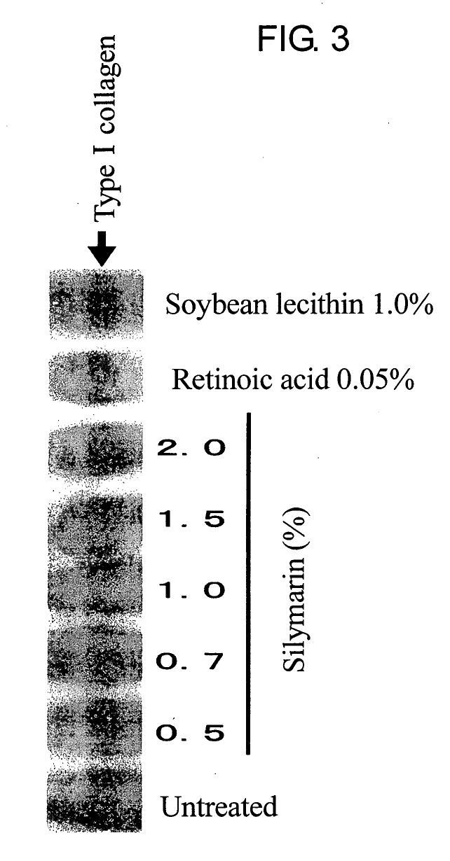

FIG. 3 shows the results measured by western blotting regarding the property of each tested substance to promote type I collagen production. Table 3 shows the numerical results obtained by image analysis with respect to full-length type I collagen (the band indicated as “Collagen” in FIG. 3).

TABLE 3

| Formulation | Concentration (%) | Relative expression level |
|---|---|---|
| Untreated control | | 1.0 |
| Soybean lecithin | 1.0 | 1.1 |
| Silymarin | 0.5 | 1.1 |
| | 0.7 | 2.7 |
| | 1.0 | 3.3 |
| | 1.5 | 2.4 |
| | 2.0 | 2.2 |
| Retinoic acid | 0.05 | 2.0 |

When three-dimensional human skin models were treated with silymarin at a concentration of 0.5%, the result was equivalent to when the untreated control was used. At concentrations of 0.7%, 1.0%, 1.5% and 2.0%, production of type I collagen was promoted by 2.7 times, 3.3 times, 2.4 times and 2.2 times, respectively. When cells were treated with retinoic acid at a concentration of 0.05% used as a positive control, the production level was promoted by 2.0 times compared with when the untreated control was used. On the other hand, when three-dimensional human skin models were treated with soybean lecithin at a concentration of 1.0% used as a negative control, the production level was equivalent to when the untreated control was used. These results show that three-dimensional human skin models treated with silymarin at a concentration of 0.7% or more promotes the production of type I collagen by 2.0 times or more.

[Evaluation of Silymarin's Property to Promote Elastin Production in a Three-Dimensional Human Skin Model]

In the same manner as described in the aforementioned evaluation of the property to promote type I collagen production in a three-dimensional human skin model, the property to promote elastin production was evaluated using a three-dimensional human skin model.

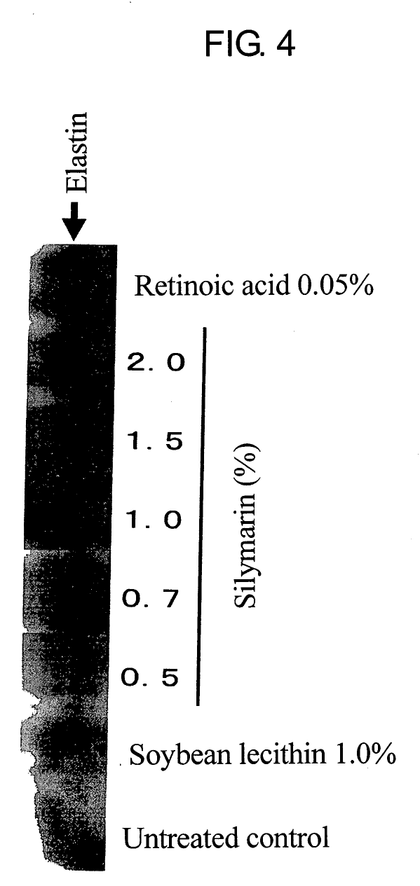

FIG. 4 shows the results measured by western blotting regarding the property of each tested substance to promote elastin production. Table 4 shows the numerical results obtained by image analysis with respect to full-length elastin (the band indicated as “Elastin” in FIG. 4).

TABLE 4

| Formulation | Concentration (%) | Relative expression level |
|---|---|---|
| Untreated control | | 1.0 |
| Soybean lecithin | 1.0 | 0.9 |
| Silymarin | 0.5 | 0.8 |
| | 0.7 | 2.5 |
| | 1.0 | 4.3 |
| | 1.5 | 3.3 |
| | 2.0 | 2.0 |
| Retinoic acid | 0.05 | 3.3 |

When three-dimensional human skin models were treated with silymarin at a concentration of 0.5%, the result was equivalent to when the untreated control was used. At concentrations of 0.7%, 1.0%, 1.5% and 2.0%, elastin production was promoted by 2.5 times, 4.3 times, 3.3 times and 2.0 times, respectively. When three-dimensional human skin models were treated with retinoic acid at a concentration of 0.05% used as a positive control, the production level was promoted by 3.3 times compared with when the untreated control was used. On the other hand, when three-dimensional human skin models were treated with soybean lecithin at a concentration of 1.0% used as a negative control, the production level was equivalent to when the untreated control was used. These results show that three-dimensional human skin models treated with silymarin at a concentration of 0.7% or more promotes the production of elastin by 2.0 times or more.

[Evaluation of Silymarin's Percutaneous Absorption Property Using Pig Skin]

Pig skin, whose absorption property has been shown to be similar to that of human skin, was used to evaluate the percutaneous absorption property of silymarin (Non-patent Literature 12). The evaluation was performed using the diffusion cell devised by Franz (Non-patent Literature 13). Yucatan mini pig skin that had been preserved at −80° C. after collection (5-month old female; manufactured by Charles River Japan) was left at room temperature for approx. 20 minutes to defrost halfway. Then, fat was removed using surgical scissors with blunt straight blades. Phosphoric acid buffer saline solution (pH 7.4) containing 0.05% sulfate kanamycin (manufactured by Wako Pure Chemical Industries) was placed in the bottom-layer receptor cell of Franz's diffusion cell, after which pig skin was placed and the upper-layer donor cell was set. The cell was placed in an incubator controlled at 37° C., and a stirrer was used to agitate the receptor cell solution. Then, 100 μl of a sample, which had been prepared by dissolving silybum marianum extract containing 70% silymarin in dipropylene glycol, was added to the donor cell and the donor cell was sealed with Parafilm. Two pieces of pig skin were evaluated for each silymarin concentration. After 24 hours, the sample left on the pig skin was removed and only the skin that had come in contact with the sample was removed using scissors. The skin was clamped using spaturas that had been heated in an 80° C. water bath, and kept clamped—and thus heated—for 20 seconds. Thereafter, tweezers were used to separate the epidermis and dermis. Next, the dermis was finely sliced using scissors and the slices were placed in a 15-ml centrifugation tube, after which 2 ml of 99.8% methanol (manufactured by Wako Pure Chemical Industries) was added and the sample was crushed by a Polytron homogenizer. The obtained substance was processed in a centrifugal separator at 10,000 G for 30 minutes to remove tissue fragments, and then filtered through a 0.45-μm membrane filter to obtain a constituent analysis sample.

Silymarin in a sample that had been prepared by dissolving silybum marianum extract containing 70% silymarin in dipropylene glycol to each silymarin concentration was used as a standard to measure the amount of silymarin permeated into the dermis of pig skin by means of high-speed liquid chromatography. The conditions of high-speed liquid chromatography are specified below. The analysis conducted under the following conditions showed peaks of silychristin, silydianin, silybin A, silybin B, isosilybin A and isosilybin B.

(High-speed Liquid Chromatography Conditions)

| | |
|---|---|
| Column: | C18 UG120, 4.6 mm to 250 mm |
| | (manufactured by Shiseido) |
| Moving phase: | Water:Methanol:Acetic acid = 65:35:5 |
| Column temperature: | 40° C. |
| Measurement wavelength: | 288 nm |
| Flow rate: | 1.0 ml/min |

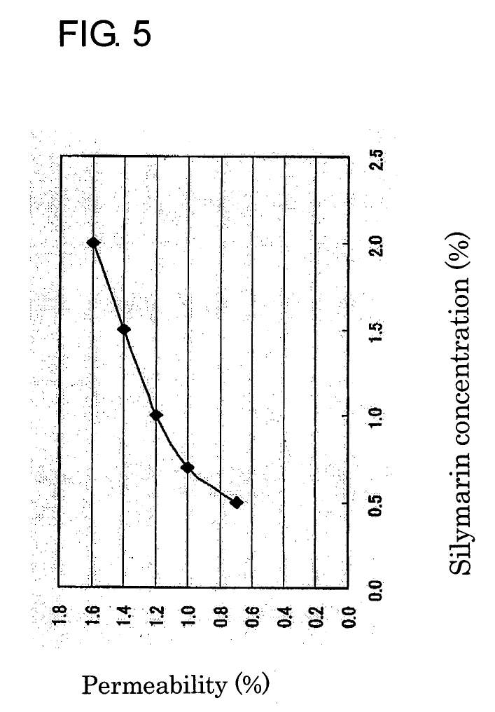

The total peak area of each constituent was calculated as silymarin concentration, and the amount of silymarin permeated into the dermis of skin pig was indicated by a percentage of the amount of silymarin in a sample that had been prepared by dissolving silybum marianum extract containing 70% silymarin in dipropylene glycol to each silymarin concentration. The results were expressed in a graph of average silymarin concentration in the dermis of two pieces of pig skin (FIG. 5). As shown, the slope corresponding to a silymarin concentration of 0.7% to 2.0% is more gradual than the slope corresponding to a silymarin concentration of 0.5% to 0.7%, suggesting that the permeability of silymarin into the dermis increases rapidly at a concentration of 0.7%.

From the evaluation results of silymarin's properties to promote type I collagen and elastin production in a three-dimensional skin model, as well as from the evaluation results of silymarin's permeability into the dermis of pig skin, it is considered that a silymarin concentration of at least 0.7% is needed for silymarin to exhibit its properties to promote type I collagen and elastin production in human skin. Accordingly, in the human tests presented below the evaluations were conducted using a cosmetic with a silymarin content of 0.7%.

[Safety Evaluation of 1% Silybum Marianum Extract Solution]

Silybum marianum has been used in foods and medicines in Europe since ancient times and its safety is well known. The property of this silybum marianum to cause skin irritation was tested.

Specifically, the human patch test method was used to evaluate the property of silybum marianum solution containing 1% silymarin to cause skin irritation. A patch, which had been treated with 1% silymarin solution, was placed on the inside of the upper arm of the subject and kept attached for 24 hours in a sealed state. Thereafter, the patch was removed and the area underneath was observed 3 hours and 24 hours later, respectively, to make judgment based on the national standard set forth by the Japanese Society for Contact Dermatitis. In the evaluation of 20 subjects, all subjects showed a negative reaction, suggesting a high safety of silymarin to the skin.

[Human Tests]

[Human Test 1] Visual Evaluation of Wrinkles

One male and 10 females complaining wrinkles were tested and the results were evaluated as specified below. The subjects used an essence prepared in accordance with recipe A by applying approx. 0.2 g of the solution on one side of their face twice a day (morning and night) for a period of 12 weeks. The subjects applied comparative example B on the other side of their face in the same manner. After the test period, improvement of wrinkles was visually evaluated by experts. Table 5 shows recipe A and comparative example B.

TABLE 5

| | | Recipe | Comparative |
|---|---|---|---|
| No. | Ingredient | A | example B |
| 1 | Dipropylene glycol | 9.000 | 9.000 |
| 2 | Glycerin | 5.000 | 5.000 |
| 3 | Betaine | 2.000 | 2.000 |
| 4 | Cargoxyvinyl polymer | 0.200 | 0.200 |
| 5 | Potassium hydroxide | 0.065 | 0.065 |
| 6 | Phytosteryl/octyldodecyl lauroyl glutamate | 1.000 | 1.000 |
| 7 | SIMULGEL NS (Note 1) | 0.500 | 0.500 |
| 8 | Silicone | 3.000 | 3.000 |
| 9 | Ethanol | 3.000 | 3.000 |
| 10 | Silymarin S (Note 2) | 1.000 | 0 |
| 11 | ID water | 75.235 | 76.235 |

Note 1: Manufactured by SEPPIC
Note 2: Manufactured by Indina

The visual evaluation found improvement in wrinkles on seven out of 11 subjects on the side on which the cosmetic based on recipe A had been applied.

[Human Test 2] Measurement of Wrinkle Depth

A test was conducted in accordance with the aforementioned method, and replicas were taken from the subjects' crow's feet before and after the test using a two-compound replica-agent scan test manufactured by Yamada Shogyo. Winkle depths at surface were determined based on measurement of the replicas using image analysis. In the image analysis of replica agent, the replica was scanned as image into a non-contact type optical 3D measuring machine (PRIMOS GFM) and the wrinkle depth at the replica surface was measured.

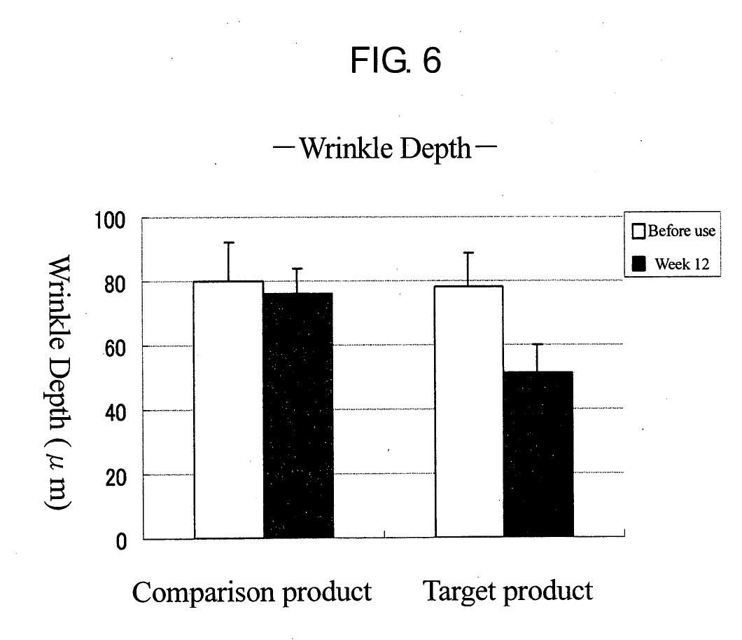

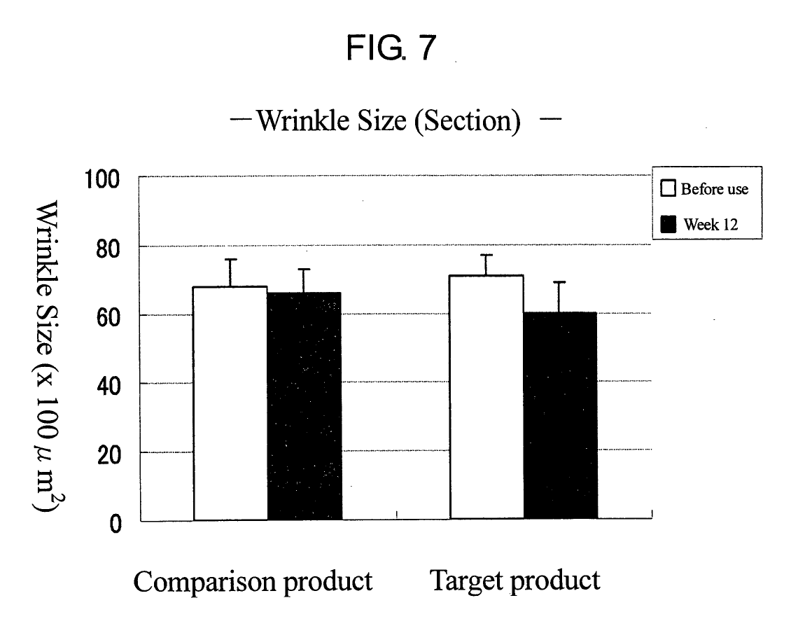

In the image analysis, wrinkle depths on the side of the face on which the cosmetic based on recipe A had been applied were significantly reduced after the test, compared with the depths before the test (the effective standard was less than 5%). FIG. 6 shows the measured wrinkle depths, while FIG. 7 shows the measured wrinkle sizes.

[Human Test 3] Skin Elasticity Test

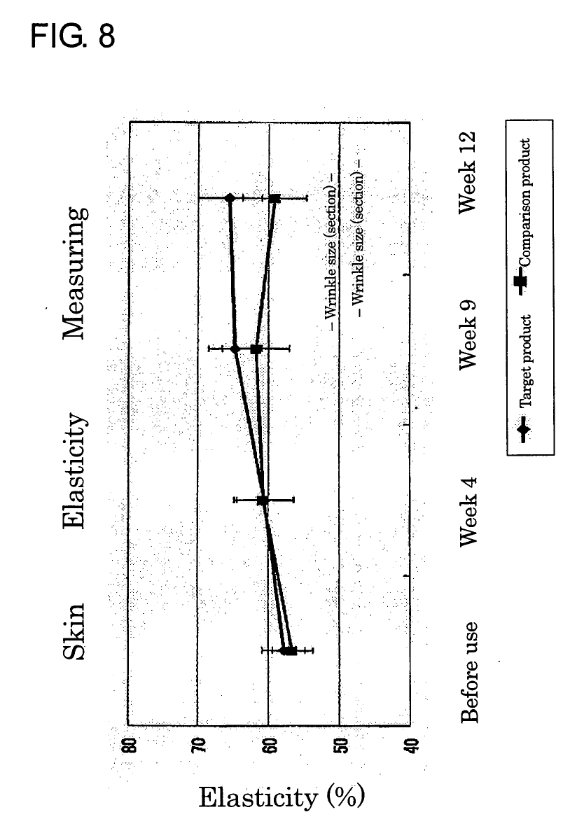

The suction elasticity of crow's feet was measured using a cutemeter (manufactured by C+K Electronic). FIG. 8 shows the results measured in the skin elasticity test.

Suction elasticity is a measuring method that provides an indicator of skin elasticity. Skin elasticity is known to decrease with age. Suction elasticity increased on both sides of the face, but the increase in suction elasticity was particularly prominent on the side of the face on which the cosmetic based on recipe A had been applied.

[Human Test 4] Measurement of Collagen Level

Collagen level was measured using SKINSKAN (an optical-fiber fluorescence spectrometer manufactured by Jobin Yvon Spec.). As an indicator, the peak areas of pepsin decomposed collagen and collagenase decomposed collagen were used.

While pepsin decomposed collagen and collagenase decomposed collagen are both known to increase by natural aging and due to exposure to light, the results found that increases of both decomposed collagens were suppressed.

[Human Test 5] Feeling Evaluation

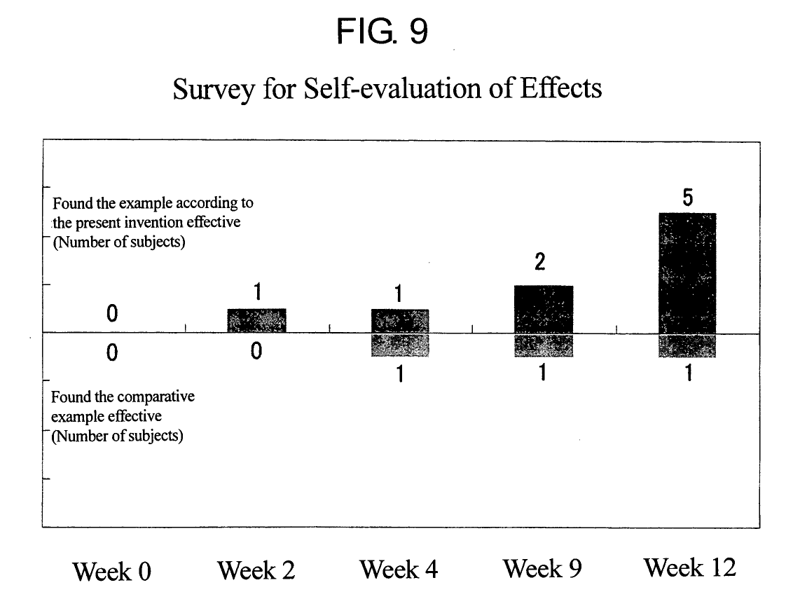

A feeling evaluation was conducted in which the subjects were asked to self-evaluate the improvement of suppleness and elasticity of their skin after 12 weeks of application. FIG. 9 shows the results of the feeling evaluation.

The evaluation found that five out of 11 subjects noticed improvement on the side of their face on which the cosmetic based on recipe A had been applied.

From the above evaluations, it is clear that the cosmetic containing silymarin is effective in improving the wrinkles (based on results of replica measurement), restoring skin elasticity, suppressing the decomposition of collagen by pepsin and collagenase, and improving the skin condition (based on self-evaluation results).

### EXAMPLES

Examples of the present invention are given below. It should be noted, however, that the present invention is not limited to these examples.

### Example 1

### Cream

A cream was manufactured based on the recipe (unit: percent by weight) shown below:

| | | |
|---|---|---|
| (1) | Stearyl alcohol | 6.0 |
| (2) | Stearic acid | 2.0 |
| (3) | Hydrogenated lanolin | 4.0 |
| (4) | Squalane | 9.0 |
| (5) | Octyl dodecanol | 10.0 |
| (6) | POE (25) cetyl alcohol ether | 3.0 |
| (7) | Glycerin monostearate | 2.0 |
| (8) | Silymarin | 0.1 |
| (9) | Preservative | As deemed appropriate |
| (10) | Aromatics | As deemed appropriate |
| (11) | 1,3-butylene glycol | 6.0 |
| (12) | PEG1500 | 4.0 |
| (13) | DI water | Remainder |

[Manufacturing Method] Constituents (1) through (10) specified above were heated to 80° C. and dissolved to create an oil phase. Constituents (11) through (13) were heated to 70° C. and dissolved to create a water phase. The water phase was gradually added to the oil phase to obtain an emulsion, and the emulsion was cooled to 40° C. under agitation, and then further agitated and cooled to 30° C. to produce a cream.

### Example 2

### Tablets

Tablets were manufactured based on the recipe (unit: percent by weight) shown below:

| | | |
|---|---|---|
| (1) | Silymarin | 20.0 |
| (2) | Milk sugar | 65.0 |
| (3) | Corn starch | 14.0 |
| (4) | Guar gum | 1.0 |

### Example 3

### Skin Milk

A skin milk was manufactured based on the recipe (unit: percent by weight) shown below:

| | | |
|---|---|---|
| (1) | Dipropylene glycol | 9.000 |
| (2) | Silybum marianum extract containing 70% silymarin | 1.000 |
| (3) | (Hydroxyethyl acrylate/acryldimethyl taurin Na) | 0.188 |
| | copolymer | |
| (4) | Squalane | 0.127 |
| (5) | Polysorbat 60 | 0.028 |
| (6) | Phytosteryl/octyldodecyl lauroyl glutamate | 1.000 |
| (7) | Glycerin | 5.000 |
| (8) | Dimethicone | 3.000 |
| (9) | DI water | 74.742 |
| (10) | Carbomer | 0.200 |
| (11) | Betaine | 2.000 |
| (12) | Ethanol | 3.000 |
| (13) | Potassium hydroxide | 0.065 |
| (14) | DI water | 0.650 |

[Manufacturing Method] Among the above constituents, (2) was added to (1) and the mixture was heated to 80° C. and dissolved. Constituents (3) through (8) were heated to 80° C. and dissolved to create an oil phase. Constituents (9) through (11) were heated to 70° C. and dissolved to create a water phase. The water phase was gradually added to the oil phase to obtain an emulsion, and the emulsion was cooled to 30° C. under agitation. Then, a mixture prepared by dissolving constituents (12) and (13) in (14) under agitation was added and the entire mixture was agitated and cooled to obtain a skin milk.

### INDUSTRIAL FIELD OF APPLICATION

Compositions containing silymarin, silymarin-containing plants or extracts from such plants as prepared in accordance with the present invention promote the production of type I collagen and elastin in the human dermis. This improves the suppleness and elasticity of the skin and keeps the skin youthful looking and free from wrinkles and sagging.

1. A composition characterized by containing silymarin and having a property to promote the production of type I collagen and/or a property to promote the production of elastin.

2. The composition as claimed in claim 1, wherein the silymarin is an extract from silymarin-containing plant and/or derived from silymarin-containing plant.

3. The composition as claimed in claim 1, which is used for preventing skin aging.

4. The composition as claimed in claim 1, which is used for external application on the skin.

5. The composition as claimed in claim 1, which is a food.

6. The composition as claimed in claim 4, which contains 0.7% to 2.0% of silymarin as an active ingredient.

7. The composition as claimed in claim 1, which is a cosmetic.

8. The composition as claimed in claim 2, which is used for preventing skin aging.

9. The composition as claimed in claim 2, which is used for external application on the skin.

10. The composition as claimed in claim 3, which is used for external application on the skin.

11. The composition as claimed in claim 2, which is a food.

12. The composition as claimed in claim 3, which is a food.

13. The composition as claimed in claim 9, which contains 0.7% to 2.0% of silymarin as an active ingredient.

14. The composition as claimed in claim 10, which contains 0.7% to 2.0% of silymarin as an active ingredient.

15. The composition as claimed in claim 2, which is a cosmetic.

16. The composition as claimed in claim 3, which is a cosmetic.

17. A method for promoting the production elastin in an extracellular matrix of a dermis, comprising administrating to a subject in need thereof an effective amount of a composition comprising silymarin.

18. The method according to claim 17, wherein the administration is oral administration.

19. The method according to claim 17, wherein the administration is topical administration.

20. The method according to claim 17, wherein the administration is such that the production of elastin becomes double.
